(19) United States
(12) Patent Application Publication
(10) Pub. No.: US 2015/0349544 A1
(43) Pub. Date: Dec. 3, 2015

(54) LIGHTING SYSTEMS FOR MEDICAL USE

(71) Applicant: Elive LLC, New Berlin, WI (US)

(72) Inventors: Thomas Lutz, Cedar Rapids, IA (US); Ernie Katris, New Berlin, WI (US); Phillip Bartoszek, New Berlin, WI (US); Peter Katris, Barrington, IL (US)

(21) Appl. No.: 14/725,960

(22) Filed: May 29, 2015

Related U.S. Application Data
(60) Provisional application No. 62/007,185, filed on Jun. 3, 2014.

(51) Int. Cl. H02J 5/00; A61B 1/24; A61C 17/06; A61C 1/08

(52) U.S. Cl. CPC H02J 5/005; A61C 1/088; A61B 1/24; A61C 17/043

(57) ABSTRACT

An induction system for use in a medical environment includes a power induction plate configured to be disposed within the medical environment, the power induction plate configured to generate an inductive field. The induction system also includes an induction coil, and a medical tool coupled to the induction coil. The power induction plate is configured to power the induction coil when the induction coil is within a predefined area surrounding the power induction plate.

### CROSS-REFERENCE TO RELATED APPLICATIONS

This application claims priority to U.S. Provisional Application No. 62/007,185, filed Jun. 3, 2014, the entire contents of which are incorporated herein by reference.

### FIELD OF THE INVENTION

The present invention relates to lighting systems for medical use, and in particular to lighting systems for the field of dentistry.

### BACKGROUND OF THE INVENTION

Visibility during a dental procedure (e.g., within a patient's mouth) is a constant challenge for dentists, due to shadowing, tight spaces, and tools getting in the way during a dental procedure. Current lighting systems use high intensity lighting, overhead flood lights, head lamps, and mirrors to help provide lighting for the dentist during a procedure. However, these current systems are large, cumbersome, and expensive, and do not always provide the desired amount of light needed during a procedure.

### SUMMARY

In accordance with one construction, an induction system for use in a medical environment includes a power induction plate configured to be disposed within the medical environment, the power induction plate configured to generate an inductive field. The induction system also includes an induction coil, and a medical tool coupled to the induction coil. The power induction plate is configured to power the induction coil when the induction coil is within a predefined area surrounding the power induction plate.

Other aspects of the invention will become apparent by consideration of the detailed description and accompanying drawings.

### BRIEF DESCRIPTION OF THE DRAWINGS

FIG. 1 is a top view of a dental bite block.

FIG. 2 is a left side view of the bite block of FIG. 1.

FIG. 3 is a right side view of the bite block of FIG. 1.

FIG. 4 is a top view of the bite block of FIG. 1, with a lighting element coupled to the bite block.

FIG. 5 is a right side view of the bite block of FIG. 1, with a saliva channel for attachment to a suction tool.

FIG. 6 is a perspective view of a pair of surgical glasses, with loupes and a bracket for attachment of a lighting element.

FIG. 7 is a perspective view of the pair of surgical glasses of FIG. 6, illustrating the lighting element being attached to the bracket.

FIG. 8 is a perspective view of a lighting system that includes the pair of surgical glasses of FIG. 6, as well as a charging station for charging additional lighting elements.

FIG. 9 is a front view of an induction system for use in a medical environment, with a lighting element that is not energized.

FIG. 10 is a front view of the induction system of FIG. 9, wherein the lighting element is energized.

FIG. 11 is a perspective view of a medical tool that includes a lighting element and a heated handle.

FIG. 12 is a perspective view of the medical tool of FIG. 11 being powered by a separate power cord.

FIG. 13 is a perspective view of the medical tool of FIG. 11, wherein the medical tool includes multiple lighting elements disposed adjacent the handle.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited.

### DETAILED DESCRIPTION

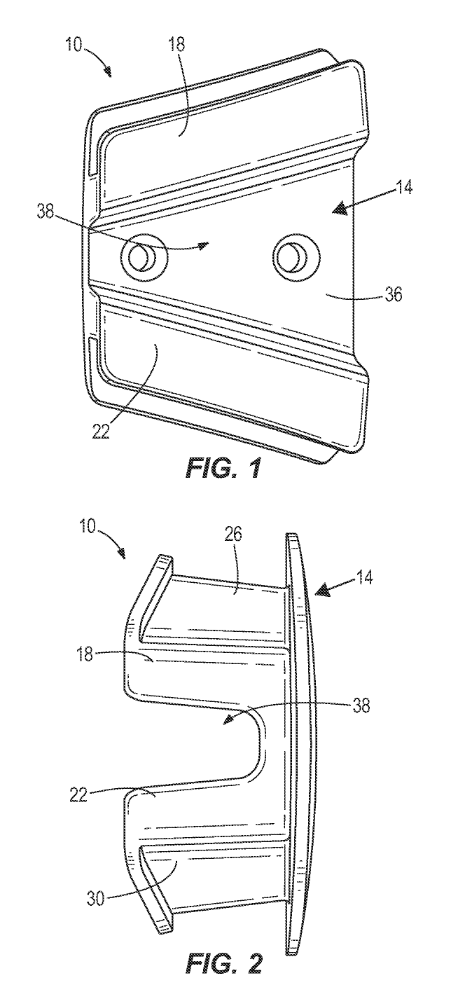

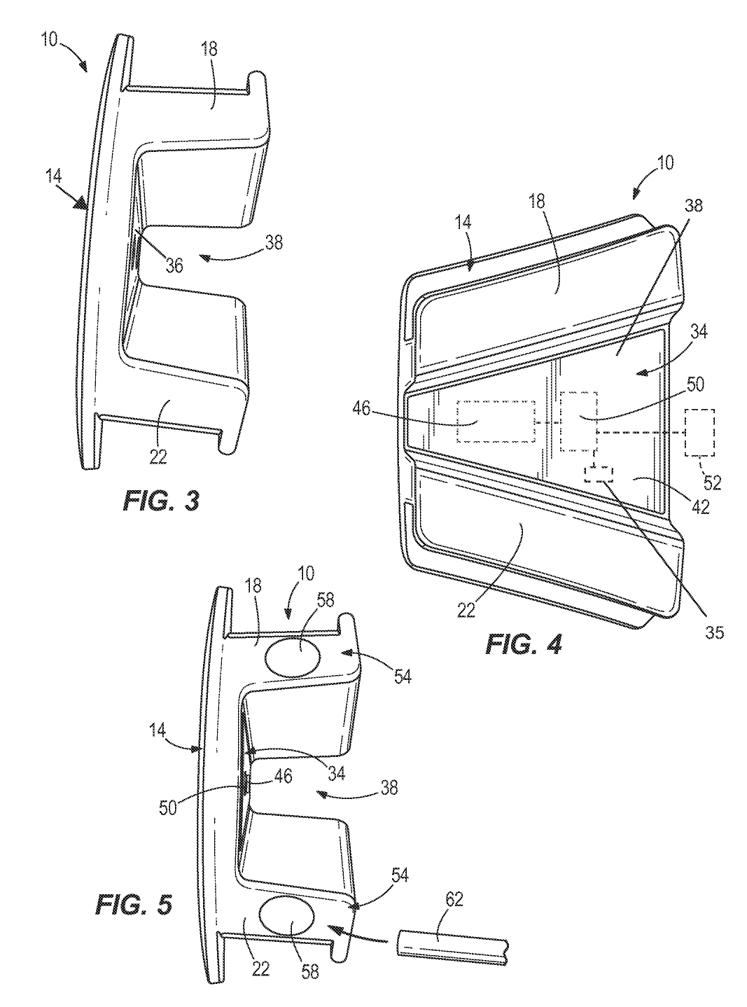

FIGS. 1-3 illustrate a dental bite block 10. The illustrated bite block 10 is a resilient, wedge-shaped structure that includes a main body portion 14, a first bite element 18 that extends from the main body portion 14, and a second bite element 22 that extends from the main body portion 14. In the illustrated construction the main body portion 14, the first bite element 18, and the second bite element 22 are integrally formed as one piece. The first and second bite elements 18, 22 are angled relative to one another at an acute angle, and include biting surfaces 26 and 30, respectively (FIG. 2). In use, the patient bites down on the biting surfaces 26, 30 to press the first and second bite elements 18, 22 toward one another. The bite elements 18, 22 provide resistance, and maintain the patient's mouth in an open position, thereby providing access for dental tools.

The illustrated bite block 10 is re-usable through sterilization in an autoclave or other suitable process. However, in some constructions the bite block 10 is disposable, and is intended only for a one-time use.

With reference to FIGS. 1-5, in some constructions the bite block 10 includes a lighting element 34 (FIGS. 4 and 5) disposed on an interior surface 36 (FIGS. 1-3) of the body portion 14 within a channel 38, the channel 38 being formed by the interior surface 36, the first bite element 18, and the second bite element 22. As illustrated in FIG. 4, the lighting element 34 includes a housing 42 (e.g., a thin housing), a battery 46 disposed within the housing, and a light-emitting element 50 (e.g., a light-emitting diode (“LED”)) disposed within the housing 42 that is powered by the battery 46. The battery 46 and light-emitting element 50 are illustrated schematically. In some constructions the battery 46 is a single, thin, button cell battery such as that used in watches (e.g., a 1-3 Volt type battery). Other constructions include different numbers and types of batteries, as well as different numbers of light-emitting elements 50. In the illustrated construction the housing 42 is a waterproof compartment. In some constructions a portion of the light-emitting element 50 is disposed outside of the housing 42.

The illustrated lighting element 34 is removable from the bite block 10. For example, in some constructions the lighting element 34 fits into the channel 38 via only a frictional hold. However, in some constructions the lighting element 34 is integrally formed as one inseparable piece with the bite block 10 (e.g., with one or more of the main body portion 14, the first bite element 18, and the second bite element 22).

In some constructions the lighting element 34 is disposed along or within other areas of the bite block 10 than within the channel 38. For example, in some constructions the lighting element 34 is formed into an area of the main body portion 14 itself

The lighting element 34 emits light from the light-emitting element 50 and illuminates the inside of a patient's mouth. The lighting element 34 provides significant amounts of light in regions within the mouth where it is otherwise difficult to generate light. In some constructions the lighting element 34 also illuminates an area outside of and surrounding the patient's mouth.

The illustrated lighting element 34 provides non-specific flood lighting within the patient's mouth. In some constructions the bite block 10 is made of transparent material to create more of an overall glow and flooding of light within the mouth. In other constructions the lighting element 34 provides more specific, directed lighting (e.g., lighting that is focused or aimed towards a particular area within a patient's mouth, such as a tooth or teeth).

In some constructions the wavelength of the light emitted from the lighting element 34 is designed specifically for a particular dental procedure. For example, in some constructions a specific wavelength (e.g., a wavelength corresponding to blue or ultraviolet light) is used to help identify and observe cavities within a patient's mouth or to better illuminate a die or stain.

In the illustrated construction, the light-emitting element 50 is turned on by a remote switch 52 (e.g., a magnetic switch, illustrated schematically in FIG. 4). In other constructions the light-emitting element 50 is turned on by a physical switch on the lighting element 34 itself. In some constructions the battery 46 is rechargeable. For example, in some constructions the battery 46 is recharged wirelessly via an inductive recharge.

While the lighting element 34 is illustrated in the context of a wedge-shaped bite block 10, in other constructions the lighting element 34 is used with (e.g., is integrally formed as one piece with, or releasably coupled to) other types of common bite blocks commonly used in the field of dentistry.

In use, a dentist or hygienist places the bite block 10, with the lighting element 34 coupled thereto, into a patient's mouth. The lighting element 34 is turned off when it is initially placed into the patient's mouth. However, in some constructions the lighting element 34 is already turned on. Once the bite block 10 is inserted into the mouth, the patient bites down on the bite elements 18, 22. The compression of the bite elements 18, 22 causes some stress on the housing 42 of the lighting element 34. However, the housing 42 is able to withstand this limited stress. Once the bite elements 18, 22 are engaged the lighting element 34 is turned on (e.g., with the switch 52). In some constructions, the act of biting on the bite block 10 actuates the lighting element 34. The lighting element 34 illuminates at least a portion of the interior of the patient's mouth. The dentist may then conduct a procedure in the mouth with sufficient lighting. Once the procedure is finished, the lighting element 34 is turned off, and the bite block 10 and lighting element 34 are removed (e.g., to be discarded or autoclaved).

With reference to FIG. 5, in some constructions the bite block 10 additionally or alternatively includes one or more saliva channels 54. As illustrated in FIG. 5, the channels 54 are formed into the first and second bite elements 18, 22. The channels 54 include a connection 58 for a suction tool 62 (e.g., hose) or adaptor to connect to the suction tool 62. The channels 54 facilitate hands-free removal of saliva during a dental procedure. Other constructions include different numbers and locations for the channels 54.

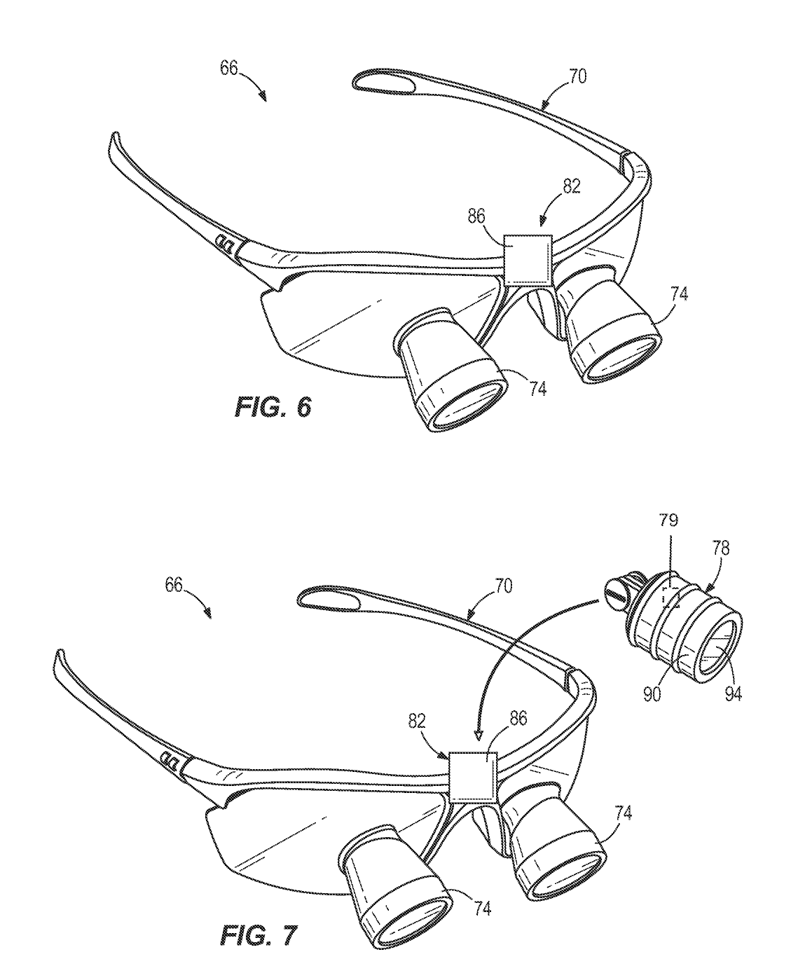

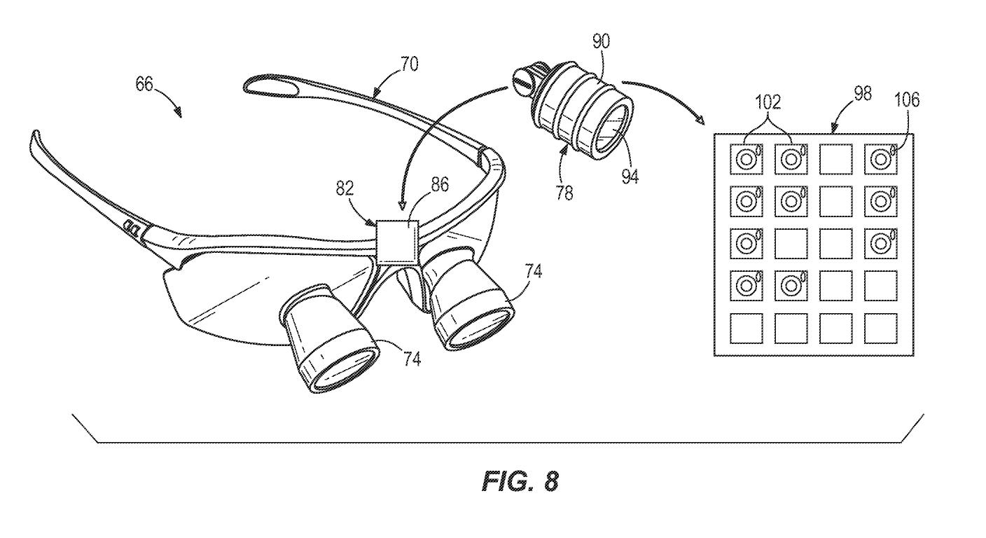

FIGS. 6-8 illustrate a head element 66 that is worn by a dentist during a procedure (e.g., during a procedure in which the bite block 10 and the lighting element 34 are also used). The head element 66 includes a pair of surgical glasses 70 worn over the dentist's ears and nose, two loupes 74 that are coupled to the glasses in front of the dentist's eyes, a removable lighting element 78 (FIGS. 7 and 8) disposed above the dentist's nose, and a mounting bracket 82 (illustrated schematically) that releasably couples the lighting element 78 to the glasses 70.

The illustrated mounting bracket 82 is a quick-connect element (e.g., allowing the lighting element 78 to snap in and out with ease), and is adjustable to fix the lighting element 78 into one or more pre-determined and reproducible positions based on the dentist's preference. For example, in some constructions the mounting bracket 82 includes an adjustment mechanism 86 (e.g., a tactile, snap, or ratchet-type mechanism) that provides an indication of the angle of the lighting element 78 relative to the glasses 70 and/or the loupes 74.

As illustrated in FIGS. 7 and 8, the lighting element 78 includes a housing 90 that houses a battery (e.g., lithium-ion, not shown) and one or more light-emitting elements 94 (e.g., an LED). The battery is configured to last approximately 2-4 hours before it needs to be recharged. This allows for a smaller battery and housing 90, and minimizes the overall weight of the lighting element 78. Other constructions include different ranges of battery life.

With reference to FIG. 8, once the battery power is exhausted in the lighting element 78, the lighting element 78 is removed and swapped with a new, identical, fully-charged lighting element 78 from a remote charging assembly 98. The new lighting element 78 is quickly and easily coupled to the mounting bracket 82, already in the user's preferred position or angle due to the adjustment mechanism 86.

The used lighting element 78 is then charged on the charging assembly 98. The illustrated charging assembly 98 is a multi-station charger having a plurality of stations 102 to receive and hold lighting elements 78. In some constructions the charging assembly 98 is a single-station charger. As illustrated in FIG. 8, each of the stations 102 includes an indicator light 106 that illuminates or changes color when lighting element 78 is disposed within the charging assembly 98 and/or indicates a state of the charge.

The lighting elements 78 are modular, cost-effective, light-weight, and efficient. While a battery life, for example, of 2-4 hours is significantly less than a full day of battery life, the use of the charging assembly 98 and the ease of coupling and de-coupling the lighting elements 78 to the glasses 70 makes the lighting elements 78 ideal for a variety of medical procedures and operating environments.

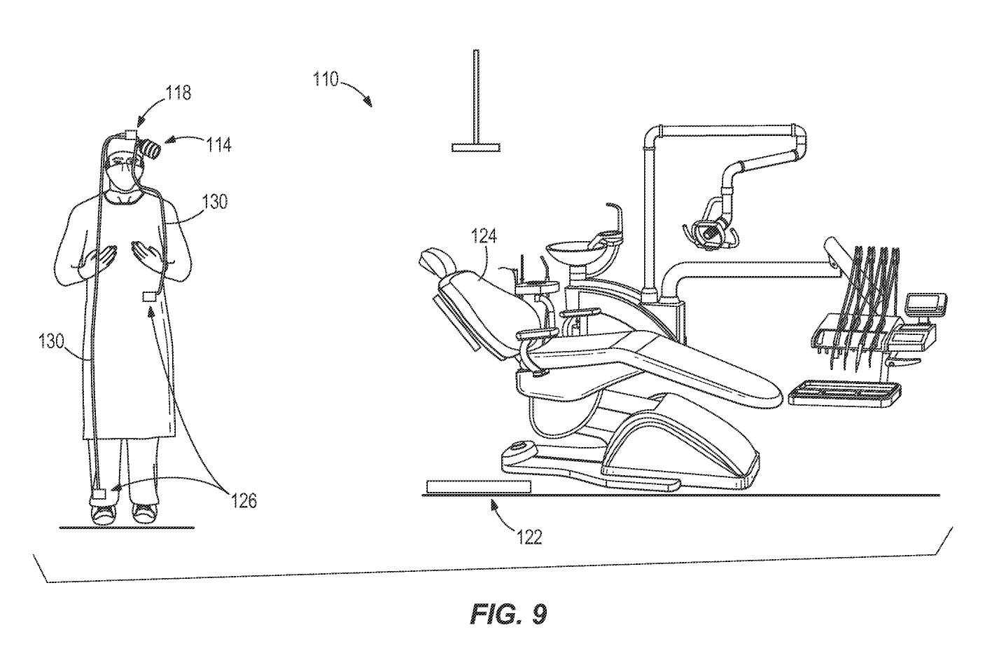

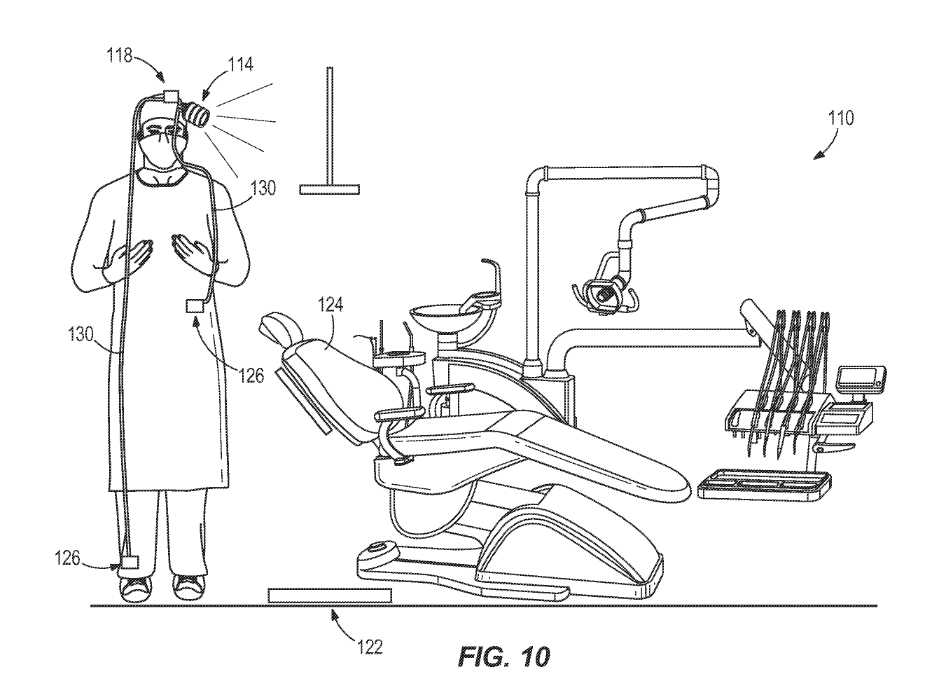

With reference to FIGS. 9 and 10, in some constructions inductive power is used to turn on one or more lighting elements or other tools. For example, FIGS. 9 and 10 illustrate an induction system 110 for use in a medical environment that includes a lighting element 114 turned off (FIG. 9) and turned on (FIG. 10) by induction. The illustrated lighting element 114 is a headlamp (e.g., with an LED) worn by a dentist. However, other constructions include different types of lighting elements. In some constructions the induction system 110 is used to turn on the lighting element 34 of the bite block 10 and/or the lighting element 78 described above (e.g., by replacing the batteries). In some constructions the induction system 110 is used to turn on medical tools other than lighting elements (e.g., brushes, drills, etc.). As noted above, in some constructions the battery 46 for the lighting element 34 of the bite block 10 is recharged wirelessly via an inductive recharge, which may be supplied for example by the power induction plate 122 of the induction system 110.

With reference to FIGS. 9 and 10, the illustrated induction system 110 includes an induction coil 118 that is worn on the dentist's head (e.g., on a strap, cap, or other head-piece). The induction coil 118 is located adjacent to the lighting element 114 and is connected with direct wiring to the lighting element 114. In some constructions the induction coil 118 and the lighting element 114 are integrally formed together as one unit or structure. In some constructions the lighting element 34 of the bite block 10 is turned on or recharged by the induction system 110, and the lighting element 34 includes an induction coil 35 (FIG. 4). In some constructions the lighting element 78 is turned on or recharged by the induction system 110, and the lighting element 78 includes an induction coil 79 (FIG. 7).

With continued reference to FIGS. 9 and 10, the lighting system 110 also includes at least one power induction plate 122 that generates an inductive field to power (e.g., turn on, charge, recharge, etc.) one or more tools. In the illustrated construction, the power induction plate or plates 122 are located within a medical environment (e.g., under each table/chair, within a floor mat, in the ceiling, etc.). As illustrated in FIG. 9, in some constructions the power induction plate 122 is adjacent a dentist chair 124. When an induction coil 118 is located within a predefined area surrounding the power induction plate 122 (e.g., within a radius of two feet, five feet, ten feet, etc.), a current is generated within the induction coil 118 that powers the lighting element 114 (e.g., turns the lighting element 114 on).

With continued reference to FIGS. 9 and 10, in some constructions one or more remote induction coils 126 (e.g., in receiver packs) are instead or additionally located on the dentist's waist, belt, near the dentist's ankles, shoes, etc. These induction coils 126 are coupled to the lighting element 114 via wires 130. Similar to the induction coil 118, when the remote induction coil or coils 126 are located close to the power induction plate 122 (e.g., within two feet, five feet, ten feet, etc.), a current is generated within the induction coil or coils 126 that powers the lighting element 114.

In some constructions the use of induction power in the induction system 110 eliminates the need for power cords and battery packs. The use of induction power (including the arrangement of the power induction plates 122 and induction coils 118, 126) also minimizes overall power requirements for an operating setting while maximizing transmission efficiencies.

In some constructions, the lighting element 114 includes an on/off switch. In some constructions, the “on” position automatically turns “off” when the dentist leaves the inductive field generated by a power induction plate 122, and/or vice versa. Thus, when the dentist moves from one chair to another continuously throughout a day he or she would not have to turn the lighting element 114 on and off Rather, when the dentist sits in a chair and approaches a patient the lighting element 114 automatically becomes energized by the power induction plate 122 beneath the chair and subsequently de-energized upon retreating from the patient. In some constructions there is an on/off foot switch or other switch that allows the power induction plate 122 to be turned on and off “hand's free” which likewise provides operation without power cords and battery packs.

While FIGS. 9 and 10 illustrate a dental operating environment, the induction system 110 is also applicable to other operating environments. In particular, the induction system 110 provides benefit for operating environments that are awkward to access, require fine dexterity skills of the professional, and benefit from reduced numbers of cords, equipment, switches, and other objects or materials that may interfere with medical personnel or patients during a procedure. Induction-powered devices are further able to reduce procedure time by alleviating issues such as large battery size, the need to recharge a battery, and reduced battery power that often occurs toward the end of a battery's life.

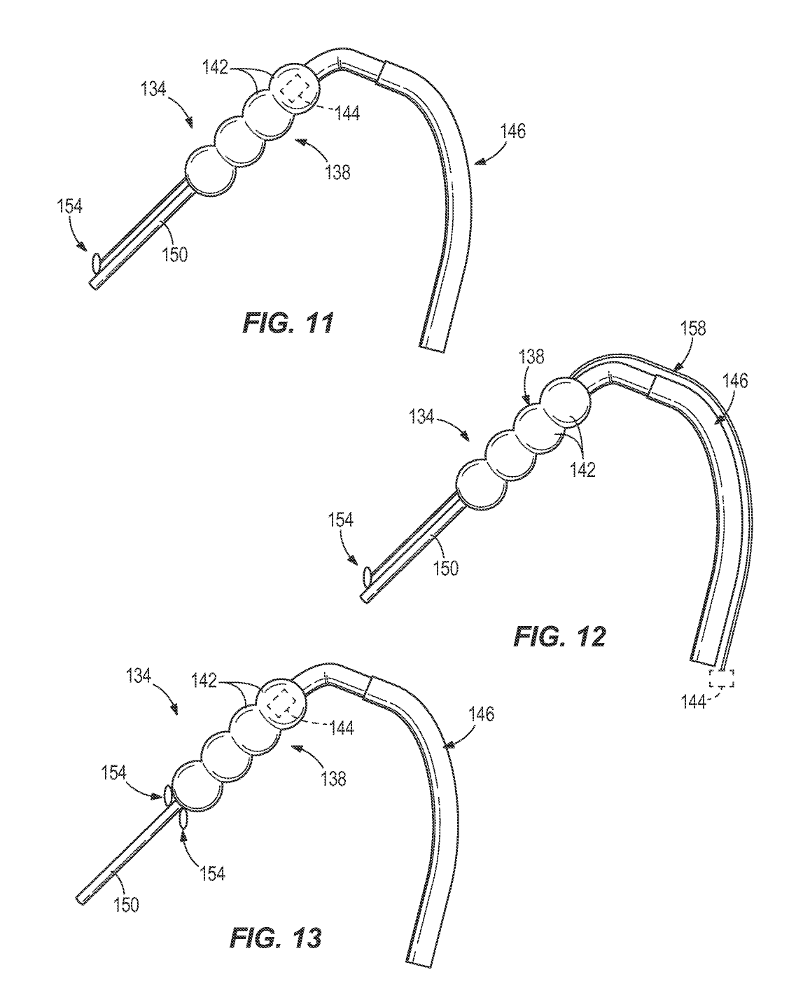

FIGS. 11-13 illustrate a medical tool 134 that is both heated and lighted. The illustrated tool 134 is a suction tool for use in a dental procedure. However, in other constructions the tool 134 is any other medical tool that benefits from both heating and lighting.

With reference to FIG. 11, the tool 134 includes a handle 138 having a plurality of ridges 142 that facilitate gripping of the tool 134. The handle 138 is heated via a power source 144 (e.g., a battery disposed within the handle 134, illustrated schematically in FIGS. 11 and 13). The ridges 142 provide an ergonomic gripping surface, such that the user's hand does not cramp or otherwise become uncomfortable after gripping the tool 134 for an extended period of time (e.g., 30 or more minutes). The heating provided in the handle 138 further alleviates discomfort, and along with the ergonomic gripping surface, inhibits the development of carpal tunnel syndrome.

The tool 134 further includes a suction line 146 that is coupled to the handle 138 and passes through or alongside the handle 138. The suction line 146 is coupled to a vacuum source (not shown) to generate a sucking action at a distal end 150 of the suction line 146. A lighting element 154 (e.g., an LED) is coupled to the distal end 150. The lighting element 154 provides illumination in a patient's mouth when the distal end 150 is placed in the patient's mouth, similar to the lighting element 34 described above. The lighting element 154 is powered by the same power source 144 (e.g., battery) as the handle 138. In other constructions the handle 138 and the lighting element 150 have separate power sources. In some constructions the tool 134 is powered by the induction system 110. For example, in some constructions the handle 134 (or other element on the tool 134) includes an induction coil as the power source 144 or as part of the power source 144, and the power source for the handle and/or lighting element 154 is an induction field generated by a power induction plate (e.g., one of the power induction plates 122 described above).

With reference to FIG. 12, in some constructions one or more of the handle 138 and the lighting element 154 are heated and powered via a wired connection 158 to a remote power source 144. With reference to FIG. 13, in some constructions one or more lighting elements 154 are disposed adjacent the handle 134 to provide maximum flood-type illumination from the tool 130.

In the illustrated construction the handle 138 is removable, and is cleaned and sterilized (e.g., via autoclave) after each use before being re-attached to the suction line 146. In the illustrated construction the lighting element or elements 154 are also removable, and are cleaned and sterilized (e.g., via autoclave) after each use before being re-attached to the suction line 146. In some constructions, the suction line 146 is disposable after a single use, such that a new suction line 146 is used with each use of the tool 134.

Although the invention has been described in detail with reference to certain preferred embodiments, variations and modifications exist within the scope and spirit of one or more independent aspects of the invention as described.

1. An induction system for use in a medical environment comprising:
a power induction plate configured to be disposed within the medical environment, the power induction plate configured to generate an inductive field;
an induction coil; and
a medical tool coupled to the induction coil;
wherein the power induction plate is configured to power the induction coil when the induction coil is within a predefined area surrounding the power induction plate.

2. The induction system of claim 1, wherein the power induction plate is disposed adjacent a dentist chair.

3. The induction system of claim 1, wherein the predefined area is a radius of ten feet.

4. The induction system of claim 1, wherein the induction coil is coupled to the medical tool via a wire, and wherein the induction coil is configured to be worn on a medical professional.

5. The induction system of claim 1, wherein the medical tool is configured to be powered off when the induction coil is outside of the predefined area.

6. The induction system of claim 1, wherein the induction coil is coupled to a light-emitting diode.

7. The induction system of claim 1, wherein the medical tool is a dental bite block.

8. The induction system of claim 7, wherein the dental bite block includes a wedge-shaped structure having a main body portion, a first bite element extending from the main body portion, and a second bite element extending from the main body portion, the first and second bite elements angled relative to one another at an acute angle and each including biting surfaces to bite down on the wedge-shaped structure, and a lighting element coupled to the wedge-shaped structure, the lighting element including a housing and a light-emitting element disposed within the housing.

9. The induction system of claim 8, wherein the housing is a battery housing, and wherein the lighting element includes a button cell battery disposed within the battery housing.

10. The induction system of claim 8, wherein the housing is disposed within a channel formed by the main body portion, the first bite element, and the second bite element, and is coupled to an interior surface on the main body portion.

11. The induction system of claim 8, wherein the lighting element is removably coupled to the wedge-shaped structure.

12. The induction system of claim 8, wherein the lighting element is configured to illuminate the inside of a patient's mouth, and wherein the light-emitting element is configured to emit a wavelength corresponding to blue or to ultraviolet light.

13. The induction system of claim 1, wherein the medical tool is a headlamp configured to be worn on a medical professional's head.

14. The induction system of claim 13, wherein the headlamp includes a mounting bracket and a plurality of modular lighting elements configured to be removably coupled to the mounting bracket.

15. The induction system of claim 14, wherein the lighting elements each include a housing and a light-emitting element disposed within the housing.

16. The induction system of claim 14, further comprising a remote charging assembly having a plurality of stations to receive and hold the modular lighting elements and to charge the modular lighting elements, each of the stations including an indicator light.

17. The modular lighting system of claim 14, wherein the headlamp includes a pair of surgical glasses having loupes, and wherein the mounting bracket is disposed between the loupes, such that the lighting element is disposed above a nose of the medical professional when the medical tool is worn by the medical professional.

18. The induction system of claim 1, wherein the medical tool includes a handle configured to be gripped by a medical professional, a suction line coupled to the handle, and a lighting element coupled to the handle, the lighting element configured to illuminate a medical working area during use of the medical tool.

19. The induction system of claim 18, wherein the handle includes a plurality of ridges, and wherein the suction line is configured to generate a sucking action at a distal end of the suction line within a patient's mouth.

20. The induction system of claim 19, wherein the lighting element is a light-emitting diode.
